(19) United States
(12) Patent Application Publication
(10) Pub. No.: US 2025/0073199 A1
(43) Pub. Date: Mar. 6, 2025

(54) Polymyxin Antibiotic Synergist and Anti-Gram-Negative Bacteria Pharmaceutical Composition

(71) Applicant: Artivila Biopharma, Xishan District Wuxi, Jiangsu, (CN)

(72) Inventors: Wei Huang, Guangdong (CN); Chunxia Hu, Guangdong (CN); Ruiqin Cui, Guangdong (CN); Huijuan Yu, Guangdong (CN)

(21) Appl. No.: 18/723,903

(22) PCT Filed: Mar. 1, 2023
(86) PCT No.: PCT/CN2023/078993

§ 371 Date: Jun. 25, 2024

(30) Foreign Application Priority Data
Jan. 18, 2022 (CN) 202210056299.7

(51) Int. Cl. A61K 31/343; A61K 38/12; A61P 31/04

(52) U.S. Cl. CPC A61K 31/343; A61K 38/12; A61P 31/04

(57) ABSTRACT

The present application discloses a polymyxin antibiotic synergist and an anti-Gram-negative bacterium pharmaceutical composition. Dronedarone or a pharmaceutically acceptable salt thereof is used as the polymyxin antibiotic synergist and combined with a polymyxin antibiotic, which can effectively improve the activity of the polymyxin antibiotic against a drug-resistant bacterium inhibit the drug resistance of the polymyxin antibiotic. Meanwhile, dronedarone can significantly reduce the dosage of the polymyxin antibiotic, which is conductive to reducing the side effects caused by the use of the drug.

### TECHNICAL FIELD

The present application relates to the technical field of biomedicine, and in particular to a polymyxin antibiotic synergist and an anti-Gram-negative bacterium pharmaceutical composition.

### BACKGROUND

According to statistics from the World Health Organization, approximately 700,000 people die from bacterial infection each year worldwide. Antibiotics are the main drugs used in the treatment of various diseases caused by bacterial infection or infection with pathogenic microorganisms. However, with the use of antibiotics, bacteria can enhance their own drug resistance through various pathways, including secretion of multiple antibiotic hydrolases (extended-spectrum β-lactamases, carbapenemases, etc.), gene mutation of drug targets, absence or displacement of membrane porins, active efflux pump systems, etc. The aforementioned pathways cause an antibiotic resistance phenomenon of the bacteria, and this phenomenon has spread worldwide, posing a serious threat to the global medical and health system. It is predicted that if no urgent action is taken, by 2030 the problem of resistance to antimicrobial drugs may push up to 24 million people into extreme poverty. The number of deaths caused by drug-resistant bacterial infections and their related diseases each year will rise sharply, reaching up to 10 million by 2050 and causing that the global economic losses can reach US$100 trillion.

In the face of increasing drug resistance, it is urgent to find and discovery new antibiotic drugs. Due to the long development cycle and low success rate of new antibiotic drugs, it is difficult to meet the demand. Antibiotic synergists, as a class of substances that have no or very low antibacterial activity but can significantly enhance the activity of existing antibiotics, have attracted widespread attention in recent years. Since the antibiotic synergists can restore the activity of antibiotics for which drug resistance have been developed, they are of great significance in alleviating the problem of drug resistance. The development of antibiotic synergists can also serve as an effective supplementary strategy for the research and development of new antibiotics, making up for their defects of low success rate.

Polymyxin antibiotics, as a class of cationic polypeptide antibiotics, can electrostatically bind to a negatively-charged lipid A on the outer membrane (OM) of a bacterium, thereby destroying the OM of the bacterium. In recent years, as the drug resistance of multidrug-resistant Gram-negative bacteria has become increasingly common, the polymyxin antibiotics have gradually become the “last line of defense” against multidrug-resistant Gram-negative bacteria. Therefore, the polymyxin antibiotics are considered to be a good target drug for the development of new antibiotic adjuvants.

### SUMMARY

An objective of the present disclosure is to overcome the aforementioned defects of the prior art and provide a polymyxin antibiotic synergist and an anti-Gram-negative bacterium pharmaceutical composition to improve the activity of polymyxin antibiotics and enhance the lethality to Gram-negative bacteria and multidrug-resistant Gram-negative bacteria.

In order to achieve the above objective, the technical solution of the present disclosure is as follows:

Use of dronedarone or a pharmaceutically acceptable salt thereof as a polymyxin antibiotic synergist.

The present disclosure further provides an anti-Gram-negative bacterium pharmaceutical composition, including a polymyxin antibiotic and a polymyxin antibiotic synergist, wherein the polymyxin antibiotic synergist includes dronedarone and/or a pharmaceutically acceptable salt thereof.

Implementation of the embodiments of the present disclosure will have the following beneficial effects:

In an embodiment of the present disclosure, dronedarone and/or a pharmaceutically acceptable salt thereof is used as a polymyxin antibiotic synergist. Dronedarone or a pharmaceutically acceptable salt thereof can inhibit the expression of genes such as arnC, arnD and the like in Gram-negative bacteria such as Klebsiella pneumoniae. The genes such as arnC, arnD and the like are exactly key genes in the synthesis and transport pathway of 4-amino-4-deoxy-L-arabinose (L-Ara4N-Lipid A) on lipid A. It has been found by proteomic analysis that the protein expression of ArnC and ArnD is also downregulated after treatment with dronedarone or a pharmaceutically acceptable salt thereof. Since the synthesis of L-Ara4N-Lipid A is the main mechanism of resistance of bacteria to polymyxins, using dronedarone or a pharmaceutically acceptable salt thereof as a synergist of polymyxins can restore the in vivo and in vitro antibacterial activities of polymyxins and inhibit the resistance of Gram-negative bacteria to polymyxins by reducing the synthesis of L-Ara4N-Lipid A.

### BRIEF DESCRIPTION OF THE DRAWINGS

To describe the technical solutions in the embodiments of the present application or in the prior art more clearly, the following briefly describes the accompanying drawings required for describing the embodiments or the prior art. Apparently, the accompanying drawings in the following description show some embodiments of the present application, and those of ordinary skills in the art may still derive other drawings from these accompanying drawings without creative efforts.

In the drawings:

FIG. 1 shows an action result of use of dronedarone combined with polymyxin B against a standard strain of Klebsiella pneumoniae (ATCC13883).

FIG. 2 shows an action result of use of dronedarone combined with polymyxin B against a clinically resistant strain of Klebsiella pneumoniae (38206).

FIG. 3 shows an action result of use of dronedarone combined with polymyxin B against a standard strain of Acinetobacter baumannii (ATCC19606).

FIG. 4 shows an action result of use of dronedarone combined with polymyxin B against a standard strain of Escherichia coli (ATCC25922).

FIG. 5 shows an action result of use of dronedarone combined with polymyxin B against a standard strain of Pseudomonas aeruginosa (ATCC27853).

FIG. 6 shows an effect of dronedarone on an overall proteome of the standard strain of Klebsiella pneumoniae (ATCC13883), wherein PB1, PB2 and PB3 are respectively control groups added with 2 μg/mL of polymyxin, and PD1, PD2 and PD3 respectively are groups added with 2 μg/mL of polymyxin B in combination with 10 μg/mL of dronedarone.

FIG. 7 shows an effect of dronedarone on the overall proteome of the standard strain of Klebsiella pneumoniae (ATCC13883).

FIG. 8 is a graph showing the survival comparison of dronedarone and/or polymyxin B used alone or in combination in the treatment of a model of BALB/c female mice infected with a polymyxin B-resistant strain (38206) of Klebsiella pneumoniae.

### DETAILED DESCRIPTION OF THE EMBODIMENTS

The following clearly and completely describes the technical solutions in the embodiments of the present application with reference to the accompanying drawings in the examples of the present application. Apparently, the described examples are merely a part rather than all of the embodiments of the present application. All other embodiments obtained by those of ordinary skills in the art based on the embodiments of the present application without creative efforts are within the claimed scope of the present application.

The present disclosure discloses use of dronedarone or a pharmaceutically acceptable salt thereof as a polymyxin antibiotic synergist against a Gram-negative bacterium. The Gram-negative bacterium can be one or two or more of Klebsiella pneumoniae, Acinetobacter baumannii, Pseudomonas aeruginosa and Escherichia coli. The polymyxin antibiotic may be selected from one or two or more of polymyxin A, polymyxin B, polymyxin C, polymyxin D and polymyxin E.

The reason why dronedarone or a pharmaceutically acceptable salt thereof can inhibit the Gram-negative bacterium is that dronedarone or a pharmaceutically acceptable salt thereof can inhibit the expression of genes such as arnC, arnD and the like in a Gram-negative bacterium such as Klebsiella pneumoniae. The genes such as arnC, arnD and the like are exactly key genes in the synthesis and transport pathway of 4-amino-4-deoxy-L-arabinose (L-Ara4N-Lipid A) on lipid A. It has been found by proteomic analysis that the protein expression of ArnC and ArnD is also downregulated after treatment with dronedarone or a pharmaceutically acceptable salt thereof. Since the synthesis of L-Ara4N-Lipid A is the main mechanism of resistance of bacteria to polymyxins, using dronedarone or a pharmaceutically acceptable salt thereof as a synergist of polymyxins can restore the in vivo and in vitro antibacterial activities of polymyxins and inhibit the resistance of Gram-negative bacteria to polymyxins by reducing the synthesis of L-Ara4N-Lipid A.

The dronedarone has a chemical name of N-[2-butyl-3-[4-[3-(dibutylamino)propoxy]benzoyl]-1-benzofuran-5-yl]methanesulfonamide, a PubChem CID number of 208898, and a specific structural formula as follows:

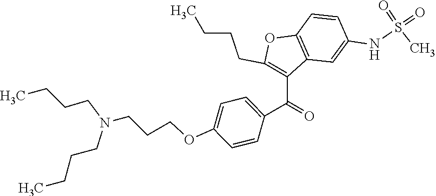 [SMILES: CCCCc1oc2ccc(NS(C)(=O)=O)cc2c1C(=O)c1ccc(OCCCN(CCCC)CCCC)cc1]

The present disclosure further provides an anti-Gram-negative bacterium pharmaceutical composition, including a polymyxin antibiotic and a polymyxin antibiotic synergist, wherein the polymyxin antibiotic synergist includes dronedarone and/or a pharmaceutically acceptable salt thereof. Specifically, dronedarone and/or a pharmaceutically acceptable salt thereof can be used in combination with a polymyxin antibiotic, or alternatively dronedarone and/or a pharmaceutically acceptable salt thereof can be mixed with a polymyxin antibiotic to prepare a formulation. The dronedarone or a pharmaceutically acceptable salt thereof can enhance the activity of the polymyxin antibiotic, and enhance the lethality of the polymyxin antibiotic against a Gram-negative bacterium and a multidrug-resistant Gram-negative bacterium.

The polymyxin antibiotic may be selected from one or two or more of polymyxin A, polymyxin B, polymyxin C, polymyxin D and polymyxin E.

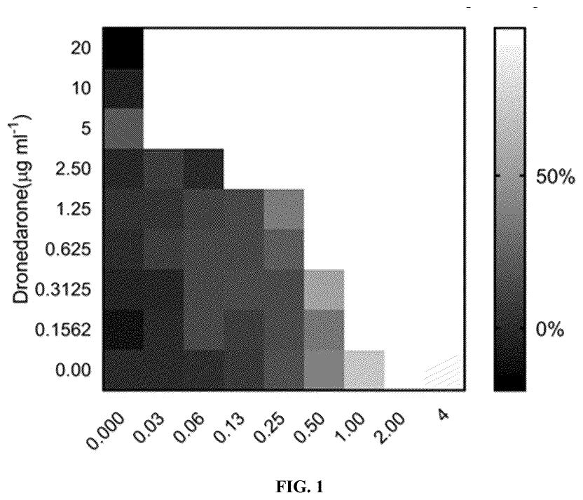

Furthermore, in the anti-Gram-negative bacterium pharmaceutical composition, the concentration of dronedarone and/or a pharmaceutically acceptable salt thereof is 0.1562 μg/mL-20 μg/mL. Preferably, the concentration of dronedarone and/or a pharmaceutically acceptable salt thereof is 10 μg/mL-20 μg/mL. Referring to FIGS. 1-5, this concentration has a significant improvement effect on all Gram-negative bacteria and drug-resistant bacteria, and meanwhile can reduce the usage amount of the polymyxin antibiotic.

In the anti-Gram-negative bacterium pharmaceutical composition, the concentration of the polymyxin antibiotic may be 0.03 μg/mL-16 μg/mL. Preferably, the concentration of the polymyxin antibiotic can be 1 μg/mL-16 μg/mL. Referring to FIGS. 1-5, when used in combination with the aforementioned dronedarone and/or a pharmaceutically acceptable salt thereof, it can be applicable to various Gram-negative bacteria.

Preferably, the mass ratio of the dronedarone or a pharmaceutically acceptable salt thereof to the polymyxin antibiotic is 19-167:1, which can exert a better pharmaceutical effect.

The following are specific examples.

In the following specific examples and test examples, the referred standard strains of Gram-negative bacteria are derived from: American Type Culture Collection (ATCC).

The referred clinical drug-resistant strains are derived from: Microbiology Laboratory, Department of Laboratory, Shenzhen People's Hospital.

A method for preparing a LB broth medium is as follows: 8 g of tryptone powder, 8 g of NaCl, and 4 g of yeast are weighed, dissolved in 800 mL of distilled water, autoclaved at 121° C. for 15 min, and stored at 4° C.

A method for preparing a PBS buffer is as follows: 8 g of NaCl, 0.2 g of KCl, 3.58 g of Na₂HPO₄·12H₂O, and 0.24 g of KH₂PO₄ are weighed, dissolved in 1,000 mL of distilled water, autoclaved at 121° C. for 15 minutes, and stored at room temperature.

### Example 1

An anti-Gram-negative bacterium pharmaceutical composition includes dronedarone and polymyxin B. The dronedarone has a chemical name of N-[2-butyl-3-[4-[3-(dibutylamino)propoxy]benzoyl]-1-benzofuran-5-yl]methanesulfonamide, a PubChem CID number of 208898, and a specific structural formula as follows:

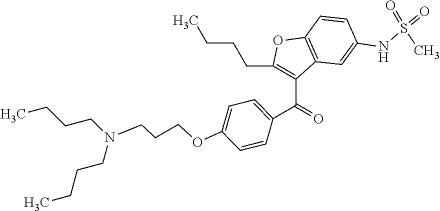 [SMILES: CCCCc1oc2ccc(NS(C)(=O)=O)cc2c1C(=O)c1ccc(OCCCN(CCCC)CCCC)cc1]

### Test Example 1

1) a standard strain of Klebsiella pneumoniae ATCC13883, a clinical resistant strain of Klebsiella pneumoniae 38206, a standard strain of Acinetobacter baumannii ATCC19606, a standard strain of Escherichia coli ATCC25922, and a standard strain of Pseudomonas aeruginosa ATCC27853 were respectively cultured at 37° C. and under shaking at 220 rpm until OD₆₀₀=0.6-0.8, where OD₆₀₀ referred to the absorbance of the solution at a wavelength of 600 nm.

2) The density of each bacterial culture with OD₆₀₀=0.6-0.8 cultured in step 1) was adjusted to OD₆₀₀=0.001 and placed in a chessboard experimental device.

3) Polymyxin B was subjected to 2-fold serial dilution along the horizontal axis, and dronedarone was subjected to 2-fold serial dilution along the vertical axis.

4) A blank group (added with a LB broth medium) and a control group (added with a LB broth medium and the standard bacterial solution cultured in step 1).

5) The final volume in each well of a 96-well plate was controlled to be 100 μL. After incubation for 18 h, each well was added with 10 μL of MTT, incubated at a condition of 37° C. with protection from light for 30 min, and then determined for the absorbance at 600 nm with a microplate reader. The bacterial inhibition rate was calculated according to the formula: bacterial inhibition rate (%)=1−(OD₆₀₀ of an experimental group−OD₆₀₀ of the blank group)/(OD₆₀₀ of the control group−OD₆₀₀ of the blank group)×100%, and a bacterial calorimetry diagram was drawn (the results were shown in FIGS. 1-5).

Referring to FIGS. 1-5, they respectively gave the relationship among the bacterial inhibition percentages of the dronedarone and the polymyxin B used in combination against the standard strain of Klebsiella pneumoniae ATCC13883, the clinical resistant strain of Klebsiella pneumoniae 38206, the standard strain of Acinetobacter baumannii ATCC19606, the standard strain of Escherichia coli ATCC25922, and the standard strain of Pseudomonas aeruginosa ATCC27853. It could be seen from FIGS. 1-5 that: when the polymyxin B was not used, the dronedarone, even at the highest concentration (reaching 20 μg/mL), has a bacterial inhibition percentage against various Gram-negative bacteria and drug-resistant bacteria of basically 0. It could be seen that the synergist of the dronedarone itself has no or very low antibacterial activity.

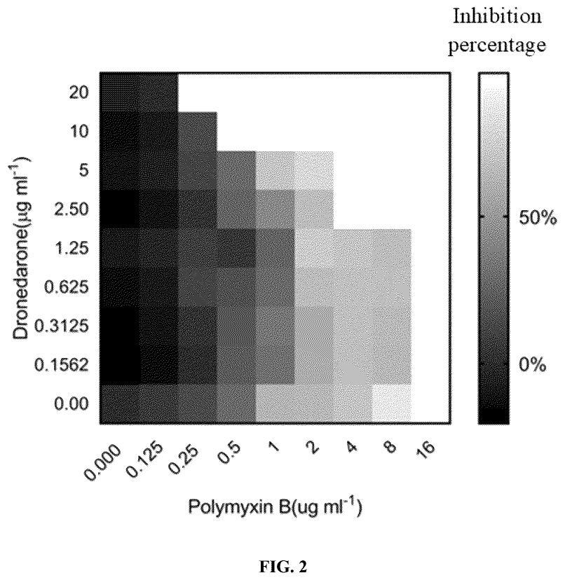

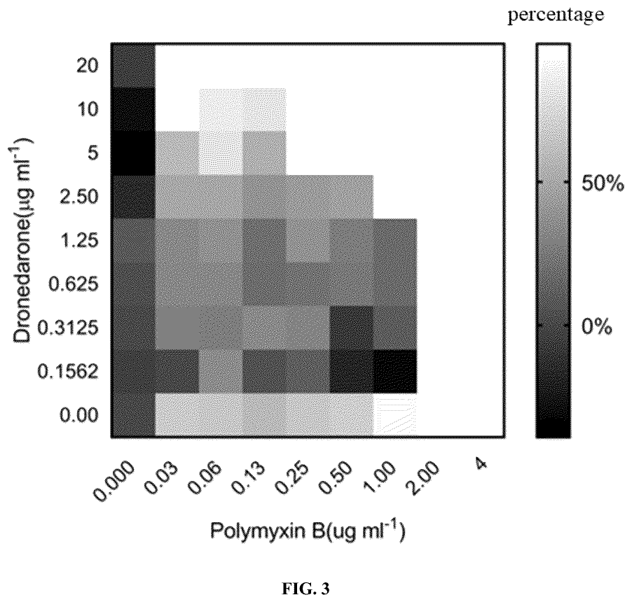

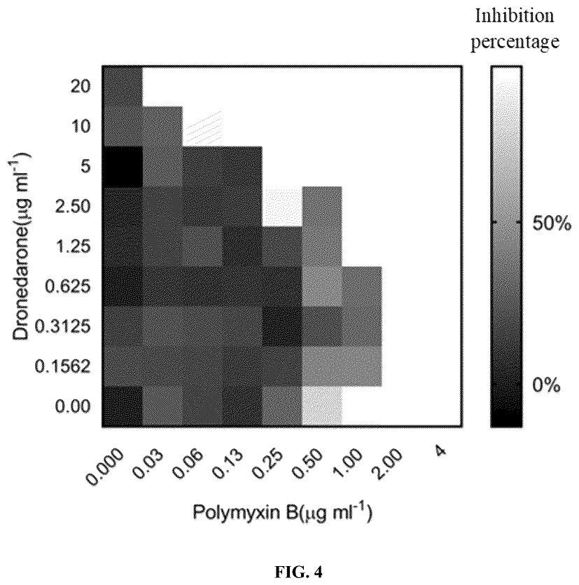

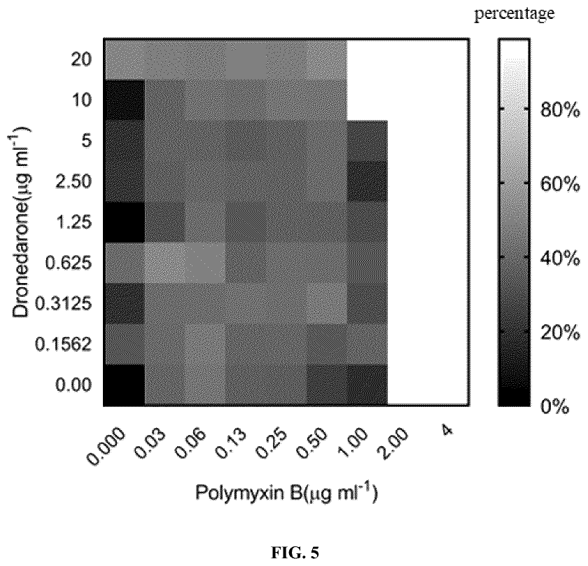

When dronedarone is not used, referring to FIG. 1, when the concentration of the polymyxin B reached 2 μg/mL, the inhibition percentage of Klebsiella pneumoniae could reach 100%; referring to FIG. 2, when the concentration of the polymyxin B reached 16 μg/mL, the inhibition percentage of resistant strain of Klebsiella pneumoniae could reach 100%; referring to FIG. 3, when the concentration of the polymyxin B reached 1 μg/mL, the inhibition percentage of Acinetobacter baumannii could reach 100%; referring to FIG. 4, when the concentration of the polymyxin B reached 1 μg/mL, the inhibition percentage of Escherichia coli could reach 100%; and referring to FIG. 5, when the concentration of the polymyxin B reached 2 μg/mL, the inhibition percentage of Pseudomonas aeruginosa could reach 100%.

Referring to FIGS. 1-5, each figure was in a step-like shape. That was, as the content of the dronedarone increased to present a white block (i.e., a bacterial inhibition percentage of 100%), the content of the polymyxin B gradually decreased. It could be seen that the dronedarone, as a synergist of the polymyxin antibiotic, could effectively improve the activity of the polymyxin and inhibit the development of drug resistance of the polymyxin. Meanwhile, the pharmaceutical composition formed from the dronedarone and the polymyxin antibiotic improved the bioavailability of the drug, could reduce the dosage of the drug, especially had a significant positive effect on the infection with Klebsiella pneumoniae/drug-resistant bacterium, Acinetobacter baumannii and Escherichia coli, etc., and was significantly better than the use of the polymyxin antibiotic alone. For the pharmaceutical composition of the present disclosure, in aspects such as the treatment and the like of infection with Klebsiella pneumoniae/drug-resistant bacteria, Acinetobacter baumannii, etc., dronedarone could significantly reduce the using dosage of polymyxin, which was conductive to reducing the side effects caused by the use of the drug and achieves a good therapeutic effect.

### Test Example 2

According to the bacterial inhibition rates, the MIC values (i.e., minimum inhibitory concentration, which referred to the minimum compound concentration that completely inhibits bacterial growth) of dronedarone and polymyxin B when used alone and the FIC index (i.e., graded inhibitory concentration) when the two were used in combination were calculated, with FIC<0.5, indicating that the combination of the polymyxin B and the compound dronedarone had a good combined effect in inhibiting bacterial growth.

The calculation equation of the FIC index was as follows:

FIC=MICab/MICa+MICba/MICb=FICa+FICb, wherein a represented the dronedarone, b represented the polymyxin B, MICab was a MIC value of a combination of the dronedarone and the polymyxin B, MICba was a MIC value of a combination of the polymyxin B and the dronedarone, MICa or MICb was a MIC value of the dronedarone or the polymyxin B, respectively, and FICa or FICb was a FIC value of the dronedarone or the polymyxin B, respectively.

According to the results of bacterial inhibition rates shown in FIG. 1, it was calculated that the MICb and MICa of the polymyxin B and the dronedarone used alone were 2 μg/mL and >20 μg/mL, respectively, and the MICba and MICab after combination of the two were 0.13 μg/mL and 2.5 μg/mL, respectively. The FIC value was <0.188, indicating that the dronedarone and the polymyxin B had significant synergistic antibacterial activity in the standard strain of Klebsiella pneumoniae (ATCC13883).

According to the results of bacterial inhibition rates shown in FIG. 2, it was calculated that the MICb and MICa of the polymyxin B and the dronedarone used alone were 16 μg/mL and >20 μg/mL, respectively, and the MICba and MICab after combination of the two were 4 μg/mL and 2.5 μg/mL, respectively. The FIC value was <0.375, indicating that the dronedarone and the polymyxin B had significant synergistic antibacterial activity in the clinical resistant strain of Klebsiella pneumoniae (38206).

According to the results of bacterial inhibition rates shown in FIG. 3, it was calculated that the MICb and MICa of the polymyxin B and the dronedarone used alone were 2 μg/mL and >20 μg/mL, respectively, and the MICba and MICab after combination of the two were 0.25 μg/mL and 5 μg/mL, respectively. The FIC value was <0.375, indicating that the dronedarone and the polymyxin B had significant synergistic antibacterial activity in the standard strain of Acinetobacter baumannii (ATCC19606).

According to the results of bacterial inhibition rates shown in FIG. 4, it was calculated that the MICb and MICa of the polymyxin B and the dronedarone used alone were 1 μg/mL and >20 μg/mL, respectively, and the MICba and MICab after combination of the two were 0.25 μg/mL and 5 μg/mL, respectively. The FIC value was <0.5, indicating that the dronedarone and the polymyxin B had synergistic antibacterial activity in the standard strain of Escherichia coli (ATCC25922).

According to the results of bacterial inhibition rates shown in FIG. 5, it was calculated that the MICb and MICa of the polymyxin B and the dronedarone used alone were 2 μg/mL and >20 μg/mL, respectively, and the MICba and MICab after combination of the two were 1 μg/mL and 10 μg/mL, respectively. The FIC value was <1, indicating that the dronedarone and the polymyxin B had no synergistic antibacterial activity in the standard strain of Pseudomonas aeruginosa (ATCC27853).

### Test Example 3

The mechanism of dronedarone in enhancing the activity of the polymyxin B in Klebsiella pneumoniae was discussed through proteomic experiments, in order to further explain the present disclosure.

Single-drug group (PB group): The standard strain of Klebsiella pneumoniae ATCC13883 was inoculated into a fresh LB broth medium added with 2 μg/mL of polymyxin at a volume ratio of 1:100, cultured at 37° C. under shaking at 220 rpm until OD₆₀₀=0.6-0.8, centrifuged at 10,000 rpm for 3 min to collect the bacteria, and then washed twice with a pre-cooled PBS buffer. The bacteria were precipitated, resuspended with four times the volume of a lysis buffer, ultrasonically treated on ice, and centrifuged under conditions of 15,000 rpm and 4° C. for 10 min. The supernatant was collected, enzymatically digested by pancreatin, and fractioned by HPLC, and then analyzed by mass spectrometry.

Combined group (PD group): The standard strain of Klebsiella pneumoniae ATCC13883 was inoculated into a fresh LB broth medium added with 2 μg/mL of polymyxin and 10 μg/mL of dronedarone at a volume ratio of 1:100, cultured at 37° C. under shaking at 220 rpm until OD₆₀₀=0.6-0.8, centrifuged at 10,000 rpm for 3 min to collect the bacteria, and then washed twice with a pre-cooled PBS buffer. The bacteria were precipitated, resuspended with four times the volume of a lysis buffer, ultrasonically treated on ice, and centrifuged under conditions of 15,000 rpm and 4° C. for 10 min. The supernatant was collected, enzymatically digested by pancreatin, and fractioned by HPLC, and then analyzed by mass spectrometry.

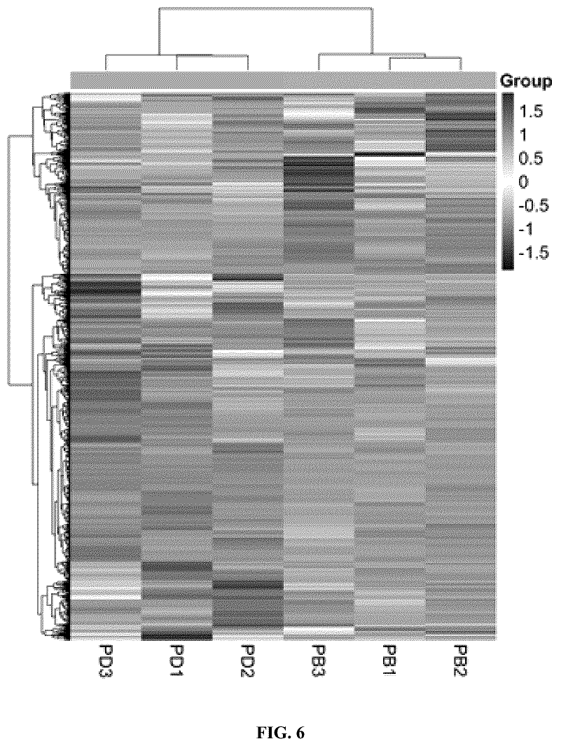

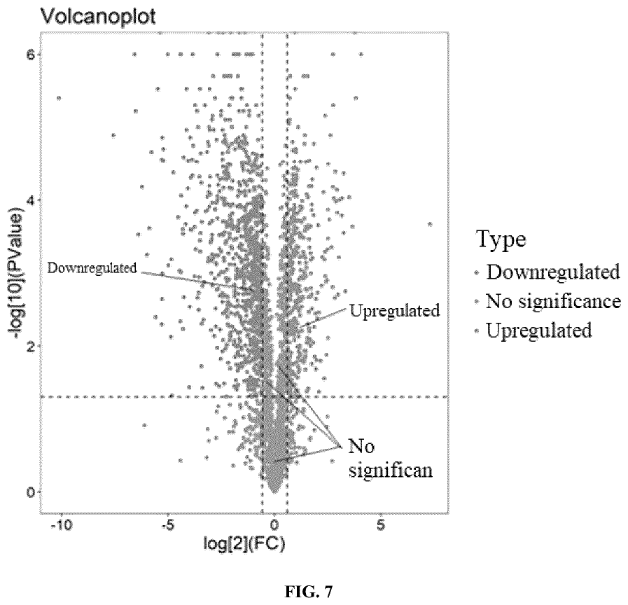

After data processing and bioinformatics analysis, the overall protein expression changes of the single-drug group and the combined group were studied. The results were shown in FIGS. 6 and 7. Referring to FIG. 7, it could be seen that: The point on the left of the left vertical dotted line was the down-regulated protein, the point on the right of the right vertical dotted line was the up-regulated protein, and the points below the two vertical dotted lines were the proteins with no significant change. Upon determination, compared with the single-drug group, in the combined group 840 proteins were down-regulated, 397 proteins were up-regulated, and the rest had no significant change, where the protein levels of ArnC and ArnD related to bacterial lipid A modification were reduced by 2.76 and 2.7 times respectively. It could be seen that in the present application, by down-regulating the expression of ArnC and ArnD proteins, the synthesis of L-Ara4N-Lipid A was reduced, the antibacterial activities of polymyxin in vitro and in vivo were restored, and the drug resistance of Gram-negative bacteria to the polymyxin was inhibited.

### Test Example 4

The dronedarone and the polymyxin B were combined to form a pharmaceutical composition, and used in combination in a mouse infection model to further illustrate the present disclosure.

1) Establishment of lung infection model: 60 BALB/c female mice aged 6-8 weeks (weighing about 20 g) were injected intrapulmonically with a lethal dose of a clinical polymyxin B-resistant strain of Klebsiella pneumoniae (38206) to establish an infection model. The concentration of the clinical polymyxin B-resistant strain of Klebsiella pneumoniae 38206 was 1.0×10⁸ CFUs per mouse;

2) Group treatment: The female mice were randomly divided into six groups (n=10 mice per group), and treated with the drug through intraperitoneal injection.

Among them, the six groups included a control group (injected with a PBS buffer), a treatment group I (injected with 0.2 mg/kg of the polymyxin B alone), a treatment group II (injected with 10 mg/kg of the dronedarone alone), a treatment group III (injected with a mixed solution of 0.2 mg/kg of the polymyxin B and 1 mg/kg of the dronedarone), a treatment group IV (injected with a mixed solution of 0.2 mg/kg of the polymyxin B and 5 mg/kg of the dronedarone), and a treatment group V (injected with a mixed solution of 0.2 mg/kg of the polymyxin B and 10 mg/kg of the dronedarone).

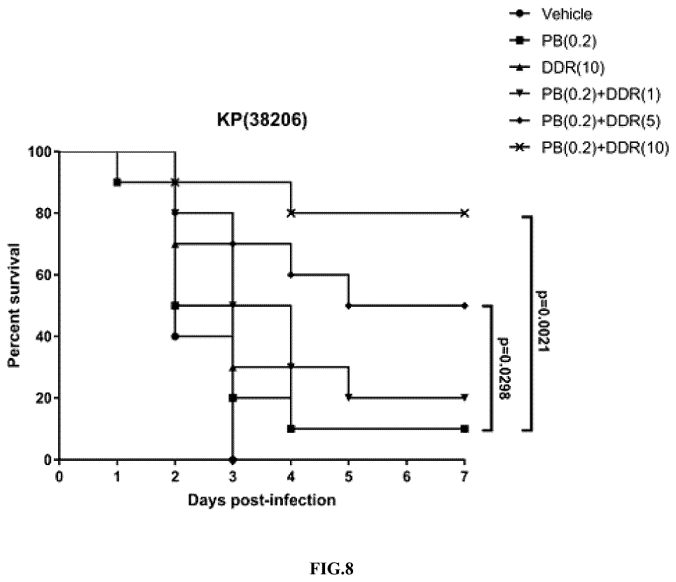

The survival rate of the female mice was observed for 7 consecutive days. The results were shown in FIG. 8. The female mice (with a survival rate of 80%) survived within 7 days after treatment with the polymyxin B (0.2 mg/kg) combined with the dronedarone (10 mg/kg), which was better than the control group injected with the polymyxin B alone (with a survival rate of 10%) or the dronedarone alone (with a survival rate of 10%). Therefore, the combination of the dronedarone and the polymyxin B significantly improved the survival rate of the mice.

1. Use of dronedarone or a pharmaceutically acceptable salt thereof as a polymyxin antibiotic synergist.

2. An anti-Gram-negative bacterium pharmaceutical composition, comprising a polymyxin antibiotic and a polymyxin antibiotic synergist, wherein the polymyxin antibiotic synergist comprises dronedarone and/or a pharmaceutically acceptable salt thereof.

3. The anti-Gram-negative bacterium pharmaceutical composition according to claim 2, wherein in the anti-Gram-negative bacterium pharmaceutical composition, a concentration of the polymyxin antibiotic synergist is 0.1562 μg/mL-20 μg/mL.

4. The anti-Gram-negative bacterium pharmaceutical composition according to claim 2, wherein in the anti-Gram-negative bacterium pharmaceutical composition, the concentration of the polymyxin antibiotic synergist is 10 μg/mL-20 μg/mL.

5. The anti-Gram-negative bacterium pharmaceutical composition according to claim 3, wherein in the anti-Gram-negative bacterium pharmaceutical composition, the concentration of the polymyxin antibiotic is 0.03 μg/mL-16 μg/mL.

6. The anti-Gram-negative bacterium pharmaceutical composition according to claim 3, wherein in the anti-Gram-negative bacterium pharmaceutical composition, the concentration of the polymyxin antibiotic is 1 μg/mL-16 μg/mL.

7. The anti-Gram-negative bacterium pharmaceutical composition according to claim 3, wherein a mass ratio of the polymyxin antibiotic synergist to the polymyxin antibiotic is 19-167:1.

8. The anti-Gram-negative bacterium pharmaceutical composition according to claim 1, wherein the polymyxin antibiotic is selected from one or two or more of polymyxin A, polymyxin B, polymyxin C, polymyxin D and polymyxin E.

9. The anti-Gram-negative bacterium pharmaceutical composition according to claim 1, wherein the Gram-negative bacterium is one or two or more of Klebsiella pneumoniae, Acinetobacter baumannii, Pseudomonas aeruginosa and Escherichia coli.

10. The anti-Gram-negative bacterium pharmaceutical composition according to claim 1, wherein the polymyxin antibiotic and the polymyxin antibiotic synergist are used in combination; or the polymyxin antibiotic and the polymyxin antibiotic synergist are mixed to prepare a formulation.
